(19) United States
(12) Patent Application Publication
(10) Pub. No.: US 2022/0249805 A1
(43) Pub. Date: Aug. 11, 2022

(54) CATHETER COMPRISING COMBINED VALVE AND SENSOR

(71) Applicant: Coloplast A/S, Humlebaek (DK)

(72) Inventor: Troels Gottfried Pedersen, Gilleleje (DK)

(21) Appl. No.: 17/620,121

(22) PCT Filed: Jun. 25, 2020
(86) PCT No.: PCT/DK2020/050193

§ 371 Date: Dec. 17, 2021

(30) Foreign Application Priority Data
Jun. 26, 2019 (DK) PA 2019 70407

(51) Int. Cl. A61M 25/00

(52) U.S. Cl. CPC A61M 25/0075; A61M 25/0043; A61M 25/0045; A61M 2205/3334; A61M 2210/1089; A61M 2205/3317; A61M 2025/0001

(57) ABSTRACT

An intermittent urinary catheterisation assembly (1) comprising an intermittent urinary catheter (3) comprising a conduit (11) extending longitudinally within the tube. The assembly further comprises a measuring system (4) configured to measure at least a pressure in the conduit and/or in a space in communication with the conduit. The measuring system comprises at least one valve element (37) in fluid communication with the conduit when the signal processing device is secured in relation to the intermittent urinary catheter. A cut-off element (41) configured to switch from a closed state to an open state. The measuring system further comprises at least one sensor element configured to measure a parameter indicative of the pressure applied to the cut-off element when the cut-off element switches from the closed state to the open state.

### FIELD OF THE INVENTION

The present invention relates to relieving urinary retention and the field of intermittent catheterization.

### BRIEF SUMMARY

Embodiments provide an intermittent urinary catheterisation assembly comprising an intermittent urinary catheter comprising a connecting portion being integral with or mounted to the non-insertable portion. The intermittent urinary catheter further comprises a conduit extending longitudinally within the tube and defining at least part of a flow path from a distal insertion end of the catheter to a proximal outlet end thereof. The intermittent urinary catheterisation assembly further comprises a measuring system configured to measure at least a pressure in the conduit and/or in a space in communication with the conduit. The measuring system comprises at least one valve element in fluid communication with the conduit when the signal processing device is secured in relation to the intermittent urinary catheter. The at least one valve element comprises a cut-off element configured to switch from a closed state disabling liquid flow in the conduit to an open state enabling liquid flow in the conduit in response to a pressure applied to the cut-off element by liquid in the conduit. The measuring system further comprises at least one sensor element configured to measure a parameter indicative of the pressure applied to the cut-off element when the cut-off element switches from the closed state to the open state. The measuring system is configured to detect a change of state of the cut-off element from the closed state to the open state. In one aspect, the signal processing device comprises a housing with an engagement mechanism for detachably securing the signal processing device in relation to the connecting portion of the intermittent urinary catheter. In another aspect, the measuring system comprising the signal processing device is securely connected to or integrated with the non-insertable portion of the catheter tube.

### BRIEF DESCRIPTION OF THE DRAWINGS

The accompanying drawings are included to provide a further understanding of embodiments and are incorporated in and constitute a part of this specification. The drawings illustrate embodiments and together with the description serve to explain principles of embodiments.

Other embodiments and many of the intended advantages of embodiments will be readily appreciated as they become better understood by reference to the following detailed description. The elements of the drawings are not necessarily to scale relative to each other. Like reference numerals designate corresponding similar parts.

FIGS. 1-3 illustrate schematic cross-sectional views of different embodiments of an intermittent urinary catheter assembly with an intermittent urinary catheter and a measuring system.

FIG. 4 illustrates a schematic cross-sectional view of an embodied intermittent urinary catheter.

### DETAILED DESCRIPTION

Intermittent catheters are typically inserted by the user him- or herself and sits only in the urethra and bladder for as long as it takes to empty the bladder—e.g. for about 5-10 minutes. Intermittent catheters are used every 4-6 hours to empty the bladder corresponding roughly to the interval that people having no urinary problems will usually go to the bathroom. Intermittent catheters are typically more rigid than indwelling catheters since they have to be inserted by the user him-/herself and since they do not need to sit in the urethra for days or weeks. An important feature for the intermittent catheter is to ease the insertion into the urethra. This is done by providing the intermittent catheter with a low frictious surface. Non-limiting examples of such are hydrophilic coated catheters which are subsequently wetted by a swelling media in order to produce a low friction surface, or oil or water-based gel which is applied to the catheter before insertion into the urethra.

Intermittent urinary catheters may be provided with a hydrophilic coating that needs to be wetted prior to use and thereby absorbs a considerable amount of liquid. Such a hydrophilic coating will provide a very lubricious surface that has very low-friction when the catheter is to be inserted. Hydrophilic coated catheters, where the coating absorbs a considerable amount of liquid for a low frictious surface (swelling degree >100%), will not be suitable for indwelling catheters, because the hydrophilic surface coating would stick inside the mucosa of the urethra if left inside the body for a longer period, due to the hydrophilic coating transforming from being highly lubricious when fully wetted to being adhesive when the hydration level of the coating is reduced.

This invention relates to intermittent urinary catheters that may be provided with a hydrophilic coating of the kind that is wetted prior to use to absorb a considerable amount of liquid and to provide a very lubricious surface.

Usually catheters used as urinary draining devices are from size 8 FR to size 18 FR. FR (or French size or Charriere (Ch)) is a standard gauge for catheters approximately corresponding to the outer circumference in mm. More accurately, the outer diameter of the catheter in mm corresponds to FR divided by 3. Thus 8 FR corresponds to a catheter with an outer diameter of 2.7 mm and 18 FR corresponds to a catheter with an outer diameter of 6 mm.

Intermittent urinary catheters typically range from CH 8-CH 16.

Intermittent urinary catheters are predominantly used for self-catheterisation by the user.

Embodiments provide an intermittent urinary catheter assembly and an intermittent urinary catheter that enable measurement of a pressure for increased utility and convenience for the user. The intermittent urinary catheter assembly and intermittent urinary catheter provide a sensor element configured to measure a parameter indicative of the pressure.

In a first aspect, embodiments provide an intermittent urinary catheterisation assembly comprising:
an intermittent urinary catheter comprising:
   a catheter tube comprising an insertable portion intended for insertion into a user's urethra, a non-insertable portion not intended for insertion into the user's urethra, and a connecting portion being integral with or mounted to the non-insertable portion; and
   a conduit extending longitudinally within the tube and defining at least part of a flow path from a distal insertion end of the catheter to a proximal outlet end thereof;
a measuring system configured to measure at least a pressure in the conduit and/or in a space in communication with the conduit, wherein the measuring system comprises:
   a signal processing device comprising a housing with an engagement mechanism for detachably securing the signal processing device in relation to the connecting portion of the catheter;
   at least one valve element in fluid communication with the conduit when the signal processing device is secured in relation to the intermittent urinary catheter, the at least one valve element comprising a cut-off element configured to switch from a closed state disabling liquid flow in the conduit to an open state enabling liquid flow in the conduit in response to a pressure applied to the cut-off element by liquid in the conduit; and
   at least one sensor element configured to measure a parameter indicative of the pressure applied to the cut-off element when the cut-off element switches from the closed state to the open state,
      wherein the measuring system is configured to detect a change of state of the cut-off element from the closed state to the open state.

In a second aspect, embodiments provide an intermittent urinary catheterisation assembly comprising:
an intermittent urinary catheter comprising:
   a catheter tube comprising an insertable portion intended for insertion into a user's urethra, a non-insertable portion not intended for insertion into the user's urethra; and
   a conduit extending longitudinally within the tube and defining at least part of a flow path from a distal insertion end of the catheter to a proximal outlet end thereof;
a measuring system securely connected to or integrated with the non-insertable portion of the catheter tube for determining at least a pressure in the conduit and/or in a space in communication with the conduit, wherein the measuring system comprises:
   a signal processing device;
   at least one valve element in fluid communication with the conduit, the at least one valve element comprising a cut-off element configured to switch from a closed state disabling liquid flow in the conduit to an open state enabling liquid flow in the conduit in response to a pressure applied to the cut-off element by liquid in the conduit; and
   at least one sensor element configured to measure a parameter indicative of the pressure applied to the cut-off element when the cut-off element switches from the closed state to the open state,
      wherein the measuring system is configured to detect if the cut-off element changes from the closed state to the open state.

Thanks to the provision of the measuring system comprising the at least one valve element and the at least one sensor element, a user of an embodied intermittent urinary catheter assembly is enabled to determine at least a pressure in the conduit and/or in a space in communication with the conduit. In embodiments, the measuring system determines the pressure in the conduit and/or in a space in communication with the conduit on the basis of the measured parameter.

The connecting portion of the intermittent urinary catheter in conjunction with the engagement mechanism in embodiments of the first aspect enables detachable securement of the signal processing device in relation to the catheter tube. Accordingly, the intermittent urinary catheter may be disposed of after each intermittent catheterisation, while the signal processing device may be used a plurality of times for more effective use of resources.

In embodiments of the first aspect, when the connecting portion and the engagement mechanism are properly secured in relation to each other, fluid communication between the conduit and the at least one valve element is enabled.

In embodiments, the connecting portion of the catheter tube and the engagement mechanism of the measuring device in embodiments of the first aspect comprise respective, mutually mating connection elements. In one embodiment, the engagement mechanism comprises resilient members configured to engage and at least partially surround, e.g., grooves at the outer circumference of the connecting portion of the catheter tube, which may provide improved tactile feedback for, e.g., physically impaired users. The engagement mechanism and connecting portion may also be embodied as, e.g., counterparts of a detachable click-lock for ease of manufacture, mutually attracting magnets for ease of attachment due to the attractive forces of the magnets working at a distance, or a frictional coupling where a protruding connecting portion of the catheter tube is forced into a receiving portion of the engagement mechanism or vice versa.

Thanks to the provision of the measuring system being securely connected to or integrated with the non-insertable portion of the catheter tube in embodiments of the second aspect, a user is able to determine the at least one fluid parameter without establishment of a connection between the catheter tube and measuring system prior to use.

In present context, the terms ‘upstream’ and ‘downstream’ refer to the flow path having a direction from the distal insertion end of the intermittent urinary catheter towards the proximal outlet end of the intermittent urinary catheter. These terms apply analogously to extensions of the flow path in fluid communication with the flow path. Accordingly, upstream refers to a flow direction towards the distal insertion end of the intermittent urinary catheter and downstream refers to a flow direction away from the distal insertion end of the intermittent urinary catheter.

When the cut-off element is in the closed state and the intermittent urinary catheter and the signal processing device are connected, liquid flow in the conduit will be disabled and the pressure within the conduit and upstream of the cut-off element will become constant.

Accordingly, the pressure applied to the cut-off element will correspond to the pressure in the conduit and/or a space in communication with the conduit such as the bladder. This allows the signal processing device to determine the pressure in the conduit and/or in a space in communication with the conduit on the basis of the measured parameter.

In embodiments, the measuring system is configured to change a state of the cut-off element from the open state to the closed state after a predetermined amount of time after detection of the change of state of the cut-off element from the closed state to the open state. This allows for repeated measurements of the pressure in the conduit and/or in a space in communication with the conduit.

In embodiments, at least one of the at least one sensor element is comprised in the signal processing device. This allows the at least one sensor element in the signal processing device to be re-used while the intermittent urinary catheter according to the first aspect is conveniently disposed of. In one embodiment, the signal processing device comprises wired or wireless connection means for connecting to- and transmitting data to another electronic device such as, e.g., a portable electronic device such as a smartphone or laptop, or a stationary computer or server. In one embodiment, the signal processing device comprises data output means as those described above.

In embodiments, the intermittent urinary catheter is disposable.

In embodiments, the intermittent urinary catheter assembly further comprises an air outlet upstream of the at least one valve element, the air outlet being configured not to allow liquid passage. The air outlet enables air to escape from the conduit and liquid propagation towards the at least one valve element in its closed state is improved until the liquid reaches the at least one valve element in the closed state. This will make the application of pressure from the liquid in the conduit to the at least one valve element more direct, and a greater accuracy of measurement may be achieved. In embodiments, the air outlet is integrated in the at least one valve element, the at least one valve element in these embodiments allowing air but not liquid to pass in the closed state.

In embodiments, the at least one valve element is configured and controllable to:
apply a force to the cut-off element to disable a liquid flow in the conduit when the signal processing device is secured in relation to the intermittent urinary catheter; and
gradually decrease the force applied to the cut-off element,
   and wherein the parameter indicative of the pressure applied to the cut-off element when the cut-off element switches from the closed state to the open state measurable by the at least one sensor element is indicative of the force applied to the cut-off element. The force applied by the pressure of the liquid in the conduit to the cut-off element will be equal to or slightly higher than the force applied to the cut-off element by the at least one valve element at the time of switching. Accordingly, the parameter indicative of the force applied to the cut-off element can readily be applied to determine a pressure in the conduit and/or in a space in communication with the conduit. Further, the parameter indicative of the force applied to the cut-off element may be straight-forward to determine as this could conveniently be a control parameter of the signal processing device.

In embodiments, at least one of the at least one sensor element is configured to measure the presence of liquid downstream of the at least one valve element for reliable detection of the cut-off element changing from the closed state to the open state.

In embodiments, the cut-off element comprises a ferromagnetic valve member and at least one electromagnet, the at least one electromagnet being configured to apply a variable force to the ferromagnetic valve member. Such embodiments allow for precise and remote variation of the force applied to the cut-off element by the at least one valve element.

In embodiments, the at least one sensor element is configured to measure an amount of electrical current conducted through the electromagnet. This allows precise measurement of the parameter indicative of the force applied to the cut-off element. The measurement may be performed indirectly in the sense of applying a known current through the electromagnet. The measurement may also be performed directly to allow measurement of, e.g., a current generated in the electromagnet caused by movement of the ferromagnetic valve member relative to the electromagnet. Such movement happen when the cut-off element switches from the closed state to the open state, and the measuring system is able to detect that the change of state of the cut-off element on the basis of the measured amount of current through the electromagnet.

In embodiments, the measuring system is configured to determine a fluid flow rate on the basis of at least the measured amount of current conducted through the electromagnet. This is enabled by correlating different vibrational modes of the ferromagnetic valve member to different fluid flow rates. The different vibrational modes may lead to measurable differences in the amount and rate of current conducted through the electromagnet on the basis of which the fluid flow rate is determined.

In embodiments, the measuring system is configured to detect said change of state of the cut-off element when the parameter or a derivate thereof exceeds a predetermined threshold value for simple detection of change of states.

In embodiments:
the valve element further comprises a restriction element upstream of the cut-off member;
the cut-off member is displaceable relative to the restriction element;
the cut-off element is configured to sealably connect to the restriction element in the closed state of the valve element. Accordingly, the closed state is achievable by applying a force to the cut-off element in the direction of the restriction element. The pressure applied from liquid at least in fluid communication with the conduit then pushes the cut-off element away from the restriction element.

In embodiments:
the at least one valve element further comprises a retention element downstream of the restriction element; wherein the retention element is configured to retain the cut-off element in the open state of the valve element. Accordingly, the cut-off element will be retained even in the open state and may be forced against the restriction element again for a further measurement.

In embodiments, the intermittent urinary catheter is configured to rest in the user's urethra for a period of time not exceeding 15 minutes. This allows the intermittent urinary catheter to comprise, e.g., coatings that do not allow extended duration of catheterisation required for indwelling catheterisation.

In embodiments, at least an outer surface of the insertable portion of the catheter tube comprises a hydrophilic surface coating. Such hydrophilic coating allows particularly convenient intermittent urinary catheterisation. In embodiments, the intermittent urinary catheterisation assembly comprises a package assembly, the package assembly enclosing the catheter tube and signal processing device and comprising an amount of liquid swelling medium for activation of the hydrophilic surface to provide the intermittent urinary catheter assembly in a ready-to-use state when the package assembly is in a storage state.

The hydrophilic coating may be provided only on the insertable part of the catheter. The hydrophilic surface coating is of the kind which, when hydrated or swelled using a swelling medium, reduces the friction on the surface area of the catheter which is intended to be inserted into the urinary channel of a user corresponding to the insertable part of the catheter.

An intermittent hydrophilic catheter differs from an indwelling catheter also in that the hydrophilic surface coating of such a catheter is not suitable for indwelling use, because the surface coating tends to stick inside the mucosa of the urethra if left inside the body for a period exceeding 5-20 minutes, due to the hydrophilic coating transforming from being highly lubricious when fully wetted (95% weight water) to being adhesive when the hydration level of the coating is reduced (<75% weight water).

In embodiments, the catheter tube is a single-lumen tube, of which the conduit constitutes the sole passage between the distal insertion end and the proximal outlet end of the intermittent urinary catheter. Such single-lumen tube catheters are generally not applicable for indwelling use because of the lack of a passage for conducting air to the balloon of an indwelling or Foley urinary catheter.

In embodiments, the signal processing device is configured to determine a fluid flow rate in the conduit on the basis of a measurement performed by the at least one sensor element.

In embodiments, at least the conduit of the intermittent urinary catheter has a pre-defined characteristic pressure drop, wherein the signal processing device is configured to determine a fluid flow rate on the basis of at least the pre-defined characteristic pressure drop and the parameter indicative of the pressure applied to the cut-off element when the cut-off element switches from the closed state to the open state. The pre-defined characteristic pressure drop is utilised to determine the characteristic pressure drop/fluid flow rate dependency and allows the fluid flow rate to be determined. The pre-defined characteristic pressure drop may be pre-determined on the basis of a reference measurement on the embodied intermittent urinary catheter, or the embodied intermittent urinary catheter may be a standardised intermittent urinary catheter wherein the pre-defined characteristic pressure drop is based on a reference measurement on another standardised intermittent urinary catheter.

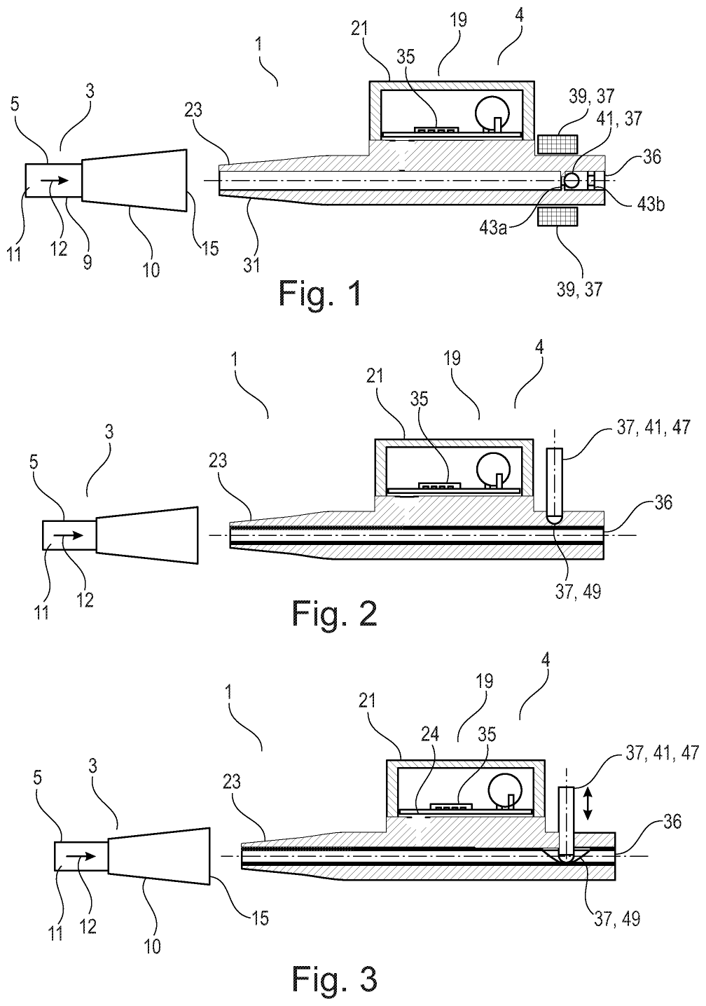

FIG. 1 illustrates a schematic cross-sectional view of an embodied intermittent urinary catheter assembly 1 with an intermittent urinary catheter 3 and a measuring system 4. The intermittent urinary catheter 3 comprises a catheter tube 5. The catheter tube 5 comprises an insertable portion 7 intended for insertion into a user's urethra, a non-insertable portion 9 not intended for insertion into the user's urethra, and a connecting portion 10 being integral with the non-insertable portion 9. The intermittent urinary catheter 3 further comprises a conduit 11 extending longitudinally within the catheter tube 5. The conduit 11 defines a flow path 12 from a distal insertion end 13 of the catheter 3 to a proximal outlet end 15 thereof. Only the non-insertable portion 9 of the catheter tube 5 and the connecting portion 10 and the proximal outlet end 15 of the intermittent urinary catheter 3 are illustrated, while the insertable portion 7 and the distal insertion end 13 of the catheter tube 5 extend outside the area illustrated in FIG. 1.

The intermittent urinary catheter assembly 1 of FIG. 1 further comprises a measuring system 4 for determining at least a pressure in the flow path 12 and/or in a space in communication with the flow path 12. In the illustrated embodiment, the measuring system 4 comprises a valve element 37 of an electronic type. An electromagnet comprising a coil 39 is wrapped around part of the signal processing device 19 to interact with a cut-off element 41 in the form of a ferromagnetic valve member 41, e.g. a ball, arranged between a restriction element 43a and a retention element 43b. By controlling the current in the coil 39, the ferromagnetic valve member 41 can be forced against the restriction element 43a in which case the valve 37 shuts off liquid flow, or it can be allowed to be pushed towards the retention element 43b which allows liquid to pass the valve element 37. The amount of current applied to the coil 39 is measured and gradually decreased to reduce the force applied to the ferromagnetic valve member 41 in the closed state of the cut-off member 41. When the ferromagnetic valve member 41 is pushed away from the retention element 43b by the pressure applied to it by liquid in communication with the conduit 5, the valve element 37 changes from a closed state (illustrated in FIG. 1) to an open state. The movement of the ferromagnetic valve member 41 relative to the coil 39 induces a measurable amount of current in the coil 39. The measuring system 4 detects the change of state from the closed state to the open state on the basis of a measurement of the induced current in the coil 39. The retention element 43b retains the ferromagnetic valve member 41 in the signal processing device 19, while allowing liquid to pass to a signal processing device outlet 36.

The amount of current applied through the coil 39 by the measuring system 4 when the change of state is detected is measured and the measured amount of current is indicative of the pressure applied to the cut-off element 41 when the cut-off element 41 switches from the closed state to the open state.

The signal processing device 19 comprises a housing 21. The housing 21 has an engagement mechanism 23 that is able to detachably secure the signal processing device 19 in relation to the connecting portion 10 of the intermittent urinary catheter 3. In the embodiment of FIG. 1, the engagement mechanism 23 is detachably secured to the connecting portion 10 by firmly inserting the protruding engagement mechanism 23 in a receiving cavity of the connecting portion 10.

In one embodiment, the intermittent urinary catheter 3 is configured to rest in the user's urethra for a period of time not exceeding 15 minutes and at least an outer surface of the insertable portion 7 of the catheter tube 5 comprises a hydrophilic surface coating. The illustrated catheter tube 5 is a single-lumen tube, of which the conduit constitutes the sole passage between the distal insertion end 13 and the proximal outlet end 15.

In embodiments, the signal processing device 19 comprises a data processing unit 35 and a power source, such as a battery or photovoltaic system.

FIGS. 2 and 3 illustrate a schematic cross-sectional view of an embodied intermittent urinary catheter assembly 1 with an intermittent urinary catheter 3 and a measuring system 4 being a variant of that of FIG. 1. In FIGS. 2 and 3 the valve element 37 is in the form of a pushing rod 47 constituting the cut-off member 41 in combination with a flexible valve membrane 49 allowing the valve element 37 to be in the open state with the flexible valve membrane 49 not restricting the flow as illustrated in FIG. 2. The flexible membrane 49 also allows the valve element 37 to be in the closed state by pushing down the pushing rod 47 to force the flexible valve membrane 49 to shut off liquid flow past the valve 37 as illustrated in FIG. 3. The pushing rod 47 is pushed down by an electric motor (not shown) driven by a continuously measured amount of current. The amount of current measured when the pushing rod 47 is pushed towards the open state by the pressure caused by liquid in communication with the conduit 5 is indicative of the pressure in the conduit 5. The pressure in the conduit is determined on the basis of the measured current. An electrical contact (not shown) is activated when the pushing rod 47 moves away from the closed state. The measuring system 4 detects a change from the closed state to the open state on the basis of the activation of the electrical contact.

FIGS. 1-3 have been illustrated and described in relation to the first aspect. Variations of the illustrated embodiments, wherein the measuring system 4 is securely connected to or integrated with the non-insertable portion 9 of the catheter tube 5 are also envisioned as in embodiments of the fourth aspect. In these variations the connecting portion 10 and the engagement mechanism 23 are exchanged with a permanent secure connection where the connecting portion 10 and the engagement mechanism 23 would otherwise detachably connect, or the catheter tube 5 and the signal processing device 19 are integrally formed where the connecting portion 10 and the engagement mechanism 23 would otherwise detachably connect.

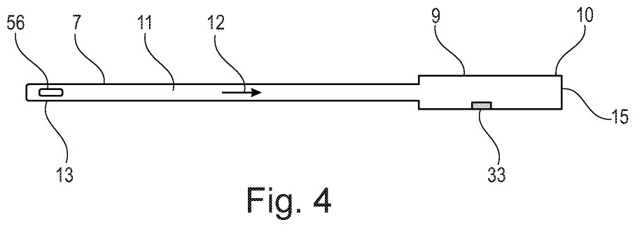

FIG. 4 illustrates a schematic cross-sectional view of an embodied intermittent urinary catheter 3. The intermittent urinary catheter 3 comprises a catheter tube 5. The catheter tube 5 comprises an insertable portion 7 intended for insertion into a user's urethra, a non-insertable portion 9 not intended for insertion into the user's urethra, and a connecting portion 10 being integral with the non-insertable portion 9. The intermittent urinary catheter 3 further comprises a conduit 11 extending longitudinally within the catheter tube 5. The conduit 11 defines a flow path 12 from a distal insertion end 13 of the catheter 3 to a proximal outlet end 15 thereof. The catheter tube 5 includes an eyelet 56 in the distal insertion end 13. The eyelet 56 allows liquid to enter the conduit 11.

1-14. (canceled)

15. An intermittent urinary catheterisation assembly comprising:
an intermittent urinary catheter comprising:
a catheter tube comprising an insertable portion intended for insertion into a user's urethra, a non-insertable portion not intended for insertion into the user's urethra, and a connecting portion being integral with or mounted to the non-insertable portion; and
a conduit extending longitudinally within the tube and defining at least part of a flow path from a distal insertion end of the catheter to a proximal outlet end thereof; and
a measuring system configured to measure at least a pressure in the conduit and/or in a space in communication with the conduit, wherein the measuring system comprises:
a signal processing device comprising a housing with an engagement mechanism for detachably securing the signal processing device in relation to the connecting portion of the catheter;
at least one valve element in fluid communication with the conduit when the signal processing device is secured in relation to the intermittent urinary catheter, the at least one valve element comprising a cut-off element configured to switch from a closed state disabling liquid flow in the conduit to an open state enabling liquid flow in the conduit in response to a pressure applied to the cut-off element by liquid in the conduit; and
at least one sensor element configured to measure a parameter indicative of the pressure applied to the cut-off element when the cut-off element switches from the closed state to the open state,
wherein the measuring system is configured to detect a change of state of the cut-off element from the closed state to the open state.

16. The intermittent urinary catheterisation assembly according to claim 15, further comprising an air outlet upstream of the at least one valve element, the air outlet being configured not to allow liquid passage.

17. The intermittent urinary catheterisation assembly according to claim 15, wherein the at least one valve element is configured and controllable to:
apply a force to the cut-off element to disable a liquid flow in the conduit when the signal processing device is secured in relation to the intermittent urinary catheter; and
gradually decrease the force applied to the cut-off element,
and wherein the parameter indicative of the pressure applied to the cut-off element when the cut-off element switches from the closed state to the open state measurable by the at least one sensor element is indicative of the force applied to the cut-off element.

18. The intermittent urinary catheterisation assembly according to claim 15, wherein at least one of the at least one sensor element is configured to measure the presence of liquid downstream of the at least one valve element.

19. The intermittent urinary assembly according to claim 15, wherein the cut-off element comprises a ferromagnetic valve member and at least one electromagnet, the at least one electromagnet being configured to apply a variable force to the ferromagnetic valve member.

20. The intermittent urinary assembly according to claim 19, wherein the at least one sensor element is configured to measure an amount of electrical current conducted through the electromagnet.

21. The intermittent urinary catheterisation assembly according to claim 15, wherein the measuring system is configured to detect said change of state of the cut-off element when the parameter or a derivate thereof exceeds a predetermined threshold value.

22. The intermittent urinary catheter assembly according to claim 15, wherein:
the valve element further comprises a restriction element upstream of the cut-off member;
the cut-off member is displaceable relative to the restriction element; and
the cut-off element is configured to sealably connect to the restriction element in the closed state of the valve element.

23. The intermittent urinary catheter assembly according to claim 8, wherein:
the at least one valve element further comprises a retention element downstream of the restriction element;
and wherein the retention element is configured to retain the cut-off element in the open state of the valve element.

24. The intermittent urinary catheterisation assembly according to claim 15, wherein the intermittent urinary catheter is configured to rest in the user's urethra for a period of time not exceeding 15 minutes.

25. The intermittent urinary catheterisation assembly according to claim 15, wherein at least an outer surface of the insertable portion of the catheter tube comprises a hydrophilic surface coating.

26. The intermittent urinary catheterisation assembly according to claim 15, wherein the catheter tube is a single-lumen tube, of which the conduit constitutes the sole passage between the distal insertion end and the proximal outlet end of the intermittent urinary catheter.

27. The intermittent urinary catheterisation assembly according to claim 15, wherein the signal processing device is configured to determine a fluid flow rate in the conduit on the basis of a measurement performed by the at least one sensor element.

28. An intermittent urinary catheterisation assembly comprising:
an intermittent urinary catheter comprising:
a catheter tube comprising an insertable portion intended for insertion into a user's urethra, a non-insertable portion not intended for insertion into the user's urethra; and
a conduit extending longitudinally within the tube and defining at least part of a flow path from a distal insertion end of the catheter to a proximal outlet end thereof; and
a measuring system securely connected to or integrated with the non-insertable portion of the catheter tube for determining at least a pressure in the conduit and/or in a space in communication with the conduit, wherein the measuring system comprises:
a signal processing device;
at least one valve element in fluid communication with the conduit, the at least one valve element comprising a cut-off element configured to switch from a closed state disabling liquid flow in the conduit to an open state enabling liquid flow in the conduit in response to a pressure applied to the cut-off element by liquid in the conduit; and
at least one sensor element configured to measure a parameter indicative of the pressure applied to the cut-off element when the cut-off element switches from the closed state to the open state,
wherein the measuring system is configured to detect if the cut-off element changes from the closed state to the open state.
